(19) United States
(12) Patent Application Publication
(10) Pub. No.: US 2013/0253294 A1
(43) Pub. Date: Sep. 26, 2013

(54) BODILY FLUID ANALYSIS DEVICE

(71) Applicant: Michael Schabbach, Frankfurt am Main (DE)

(72) Inventor: Michael Schabbach, Frankfurt am Main (DE)

(73) Assignee: SANOFI-AVENTIS DEUTSCHLAND GMBH, Frankfurt am Main (DE)

(21) Appl. No.: 13/992,333

(22) PCT Filed: Dec. 16, 2011
(86) PCT No.: PCT/EP11/73082

§ 371 Date: Jun. 7, 2013

(30) Foreign Application Priority Data
Dec. 17, 2010 (EP) 10195683.7

(51) Int. Cl. A61B 5/157; A61B 5/00; A61B 5/1468

(52) U.S. Cl. CPC A61B 5/157; A61B 5/1468; A61B 5/742
USPC 600/345

(57) ABSTRACT

An apparatus for analysing a bodily fluid sample, comprising a substrate having supported thereon: bodily fluid collecting means configured to collect a bodily fluid from the surface of a body part placed adjacent to the bodily fluid collecting means; means for analysing the collected bodily fluid sample; and means for displaying an indication of a result of the analysis.

### CROSS REFERENCE TO RELATED APPLICATIONS

The present application is a U.S. National Phase Application pursuant to 35 U.S.C. §371 of International Application No. PCT/EP2011/073082 filed Dec. 16, 2011, which claims priority to European Patent Application No. 10195683.7 filed Dec. 17, 2010. The entire disclosure contents of these applications are herewith incorporated by reference into the present application.

### FIELD OF DISCLOSURE

This invention relates to a bodily fluid analysis device.

### BACKGROUND

Many people are required to perform regular tests on their blood or other bodily fluid in order to monitor a disease and adjust medication appropriately. For example, diabetes sufferers may be provided with quantities of insulin, for instance by injection, sometimes a number of times daily. The quantity of insulin that is appropriate depends on the person's blood glucose level, so blood glucose level measurement can also occur a number of times daily.

Blood glucose level measurement typically is a multi stage process requiring a number of separate pieces of equipment. A user may be required to carry around a bulky electronic blood glucose meter as well as a supply of test strips and lancet. After lancing the skin to elicit a blood sample, a user must transfer the blood to the test strip, activate the meter and present or insert the test strip into the meter in order to obtain a measurement.

### SUMMARY

A first aspect provides an apparatus for analysing a bodily fluid sample, comprising a substrate having supported thereon:
bodily fluid collecting means configured to collect a bodily fluid from the surface of a body part placed adjacent to the bodily fluid collecting means;
means for analysing the collected bodily fluid sample; and
means for displaying an indication of a result of the analysis.

The analysis of the bodily fluid sample may comprise measuring a property of the bodily fluid and the displaying of an indication of the result of the analysis may comprise displaying the result of the measuring.

The means for analysing the collected bodily fluid sample may comprise a controller and at least one pair of electrodes connected to the controller. The at least one pair of electrodes may be integral with the means for collecting a bodily fluid sample.

The means for analysing the collected bodily fluid sample may be configured to analyse at least one electrical signal received via a first pair of the at least one pair of electrodes. The apparatus may be configured to activate the means for analysing in response to a predetermined electrical signal received via a second pair of the at least one pair of electrodes.

The apparatus may also be configured to activate the means for displaying in response to a predetermined electrical signal received via the second pair of electrodes. Alternatively, the apparatus may be configured to activate the means for displaying in response to a predetermined electrical signal received via a third pair of the at least one pair of electrodes.

The second pair of electrodes may be located further from a bodily fluid collecting end of the means for collecting than the first pair of electrodes. The third pair of electrodes may be located closer to a bodily fluid collecting end of the means for collecting than the first pair of electrodes.

The apparatus may further comprise an adjustable calibration resistor. The bodily fluid collecting means may comprise an absorbent material.

A second aspect provides a method for analysing a bodily fluid sample, comprising:
placing a body part adjacent to bodily fluid collecting means of a bodily fluid analysis device, wherein the bodily fluid collecting means are configured to collect a bodily fluid from the surface of the body part;
analysing the collected bodily fluid sample;
displaying an indication of a result of the analysis.

A third aspect provides an apparatus, the apparatus comprising:
a substrate, the substrate having supported thereon:
a collector for collecting a bodily fluid from the surface of a body part placed adjacent to the collector;
an analyser for analysing the collected bodily fluid sample; and
a display for displaying an indication of a result of the analysis.

### BRIEF DESCRIPTION OF THE DRAWINGS

Embodiments will now be described, by way of example only, with reference to the accompanying drawings, in which:

FIG. 1 shows a cutaway view of a bodily fluid analysis device;

FIG. 2 shows a schematic view of the bodily fluid analysis device of FIG. 1;

FIGS. 3A-C show the bodily fluid analysis device of FIGS. 1 and 2 in an exemplary operation;

FIG. 4 is a flow chart illustrating an exemplary operation of the bodily fluid analysis device of FIGS. 1 and 2;

FIG. 5 shows a schematic view of a second embodiment of a bodily fluid analysis device;

FIGS. 6A-C show the bodily fluid analysis device of FIG. 5 in an exemplary operation;

FIG. 7 is a flow chart illustrating an exemplary operation of the bodily fluid analysis device of FIG. 5; and

FIG. 8 shows a schematic view of a third embodiment of a bodily fluid analysis device.

### DETAILED DESCRIPTION

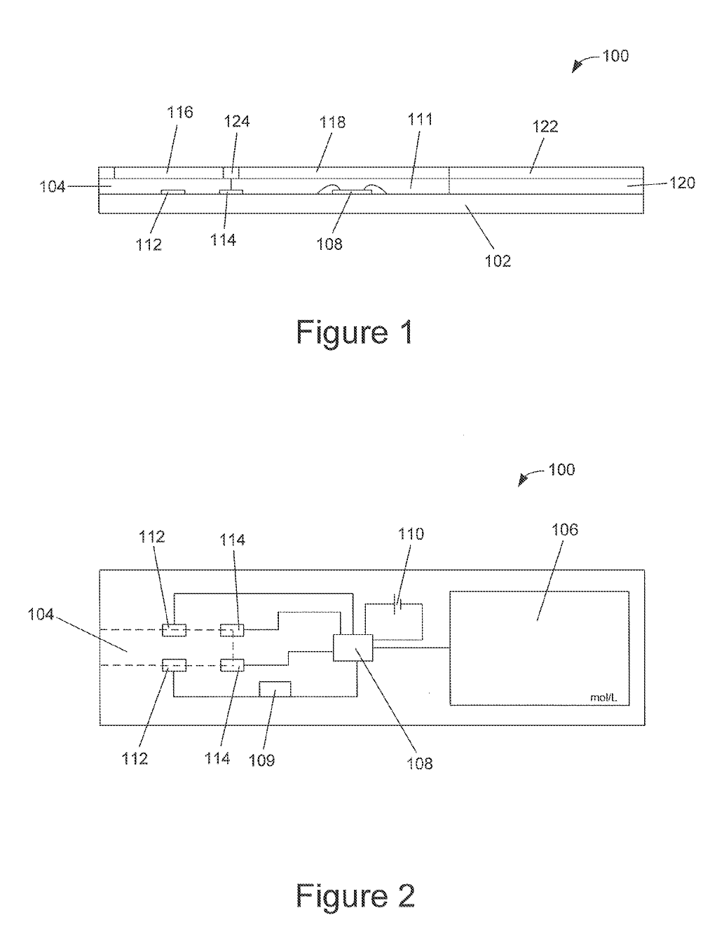

Referring firstly to FIGS. 1 and 2, a cutaway view and a schematic view respectively of a bodily fluid analysis device 100 are shown. The analysis device 100 has a layered structure. The bottom layer comprises a substrate 102 in the form of a relatively thin, elongate sheet material. Formed on top of the substrate 102 at a first end of the substrate 102 is an absorbent material 104. Formed on top of the substrate 102 at a second end, opposed to the first end, of the substrate 102 is a display 106. Electronic components are provided on top of the substrate 102 at and around the centre of the device 100. The electronics include a controller 108, a printed resistor 109 and batteries 110. The electronics are covered by a spacer glue layer 111.

The controller 108 is connected to at least one pair of electrodes 112, 114, which are also formed on top of the substrate 102. The device 100 has measurement electrodes 112 and activation electrodes 114. Both the measurement electrodes 112 and the activation electrodes 114 overlap with the absorbent material 104. The absorbent material 104 has an exposed end located at an extremity of the device 100. The measurement electrodes 112 are located closer to this exposed end of the absorbent material 104 than the activation electrodes 114.

The top layer of the device 100 (visible only in FIG. 1) comprises at least one cover which protects the components formed on top of the substrate 102. A first cover plate 116 overlies the absorbent material 104 and a second cover plate 118 overlies the electronics and spacer glue 111. The display 106 may be a Liquid Crystal Display (LCD), comprising an LCD fill 120 and a LCD cover plate 122. A capillary vent 124 is defined at the point where the first cover plate 116 and the second cover plate 118 meet.

The controller 108 is connected to the LCD fill 120 of the display 106 and to the measurement electrodes 112 and the activation electrodes 114. These connections may be made via conductive paths formed on the substrate 102. The controller 108 is configured to receive signals from the measurement electrodes 112 and the activation electrodes 114 and to send control signals to the display 106. The measurement electrodes 112 and the activation electrodes 114 may be made of any conductive material, for example carbon or a metal.

The absorbent material 104 may be located at a first end of the substrate 102 such that one end of the absorbent material 104 is exposed. The end of the absorbent material 104 which is exposed may be made more noticeable to a user of the device 100 by the addition of colour, texture or other markings, a notch or indentation or by protruding a short distance beyond the substrate 102 and first cover plate 116. The absorbent material 104 may be a wicking material having a capillary structure. Fluid applied to the exposed end of the absorbent material 104 is drawn into the body of the absorbent material 104 by capillary action. The capillary vent 124 may facilitate the capillary action by allowing air within the absorbent material 104 to be displaced by the fluid.

The absorbent material 104 may contain a chemical substance such as an enzyme. The absorbent material 104 may be doped with the chemical substance during manufacture. When bodily fluid is absorbed into the material 104, it reacts with the chemical substance to create an electrical signal. The chemical substance present in the absorbent material 104 may be selected to react with specific target substance in the bodily fluid or to catalyze a reaction involving a target substance in the bodily fluid. For example, the target substance may be blood glucose. The blood glucose may react with an enzyme in the absorbent material 104 which is subsequently reoxidised to produce electrical charge. A mediator may be in involved in the reoxidation of the enzyme. The electrical charge produced may then flow as a current through attached electrodes. The target substance may be another component of blood, for example glycated haemoglobin (HbA1c) or a ketone.

The chemical substance may be present throughout the absorbent material 104 or it may only be present in a portion of the absorbent material 104. The absorbent material 104 may occupy the entire width of the substrate 102 or it may occupy only a portion of the width of the substrate 102. In the embodiment of FIG. 2, the absorbent material 104 occupies only a portion of the whole width of the substrate 102 and is centrally located as is indicated by the dashed line. The remaining material at this first end of the device 100 is a non absorbent material, such as a plastic. Alternatively the substrate 102 may be of greater thickness so as to form a recess for the absorbent material 104. The substrate 102 may be a plastic material or any other suitable material such as silicon.

In some embodiments, the absorbent material 104 is configured to absorb a predetermined amount of fluid. Depending on the bodily fluid which is being absorbed, the property of the fluid which is to be measured and the method by which the measurement is to be taken, the volume of fluid present in the absorbent material 104 may affect the measurement. Configuration of the absorbent material 104 to absorb a predetermined amount of fluid helps to improve measurement accuracy.

The controller 108 may be a microcontroller or integrated circuit of any suitable type. The controller 108 may store software or algorithms in flash memory (not shown) and may have volatile memory such as RAM (not shown) for executing the software. The spacer glue layer 111 covering the controller 108 and other electronics ensures that no circuit crossing or shorting occurs and prevents the electronics from becoming dislodged or disconnected. The controller 108 is connected to each of the measurement electrodes 112 in order to measure a property of the bodily fluid sample absorbed in the absorbent material 104.

The batteries 110 are configured to provide power to the controller 108 and the display 106. In some embodiments, the batteries have a energy storage capacity which is sufficient only to power the controller 108 to compute a single measurement and to power the display 106 for a reasonable time, e.g. a few minutes. As the device 100 requires only a small amount of energy, the batteries 110 may be replaced with another source of power such as a capacitor able to collect energy from radio frequency signals emitted by a mobile transmitting device such as a mobile phone. Thus a user of the device 100 may be able to power or charge the device 100 using an application on their mobile phone or the device 100 may charge by being in close proximity to the user's mobile phone for a short time. The device 100 may also be able to collect energy from ambient electromagnetic radiation. In some embodiments, the batteries 110 are replaced with means for collecting energy from a user of the device 100. For example the device 100 may have an area which the user compresses between their fingers to impart energy to the device 100. Alternatively the device 100 may contain both batteries 110 and other means for collecting energy.

The measurement electrodes 112 and the activation electrodes 114 are disposed on the substrate 102 and overlap with at least a portion of the absorbent material 104. Each of the measurement electrodes 112 and the activation electrodes 114 may comprise a pair of electrodes spaced apart. The space between the electrodes is at least partially occupied by absorbent material 104. The measurement electrodes 112 are configured to transmit electrical signals generated by the reaction of bodily fluid with a chemical substance present in the absorbent material 104 to the controller 108 so that a property of the bodily fluid can be measured. This property may be a concentration of a specific substance present in the bodily fluid. The activation electrodes 114 are also configured to pass electrical signals generated in the absorbent material 104 to the controller 108. These signals are used as a trigger for activation of the controller 108 and the display 106. For example, the controller 108 may be configured to detect only signals above a certain threshold which are received from the activation electrodes 114. When a signal above the threshold is detected, the controller 108 and display 106 are activated.

The resistor 109 is disposed on a conductive path linking the controller 108 to one of the measurement electrodes 112. This allows the electrical resistance of the circuit path formed by the controller 108, measurement electrodes 112 and absorbent material 104 to be predetermined. The controller 108 may rely on having an accurate value for the resistance of this circuit path in order to produce an accurate measurement. The resistor 109 may be a printed circuit resistor.

In alternative embodiments, the resistor 109 is disposed on a conductive path that begins and ends at the controller 108 and does not incorporate any of the electrodes 112, 114. The resistor may be used for calibration, for example to adjust a lot to lot manufacturing accuracy of the device 100. For example, after determining a calibration value of a lot of test strips during production, the resistance of the resistor 109 may be adjusted by laser cutting.

The resistor 109 may be printed on the substrate 102 with a carbon containing ink, for example. After determining a calibration value of the lot of test strips during production, the resistance of the resistor 109 may be adjusted by modifying the printing process of resistor 109. The resistance of resistor 109 may be used during use of device 100 as a calibration value to adjust a measurement result.

In further example embodiments, a capacitor is used for storing a calibration value on the device 100 instead of a resistor. Modifying the capacitor may be done by laser cutting or the printing process during production.

In further example embodiments, a calibration value may be stored in a non-volatile memory of the device during production, for example in the flash memory. Thus, no dedicated resistor or capacitor may be needed for calibration.

The batteries 110 are disposed on a conductive path which begins and ends at the controller 108.

The display 106 may be a numerical display configured to display three digits. In some other embodiments the display 106 may be configured to display more than three digits. The best choice of display depends on the range and accuracy of the measured property required. In some embodiments, the display 106 may be configured to display a decimal point before or after the first or second digit. Alternatively the decimal point may be in a fixed location, for example after the second digit. Each digit of the display 106 may comprise a seven-segment display capable of displaying the numbers 0 to 9. Only a single connection between the controller 108 and the display 106 is shown, however there may be several individual connections. There may be a connection between the controller 108 and the display 106 for each digit, or for each segment of each digit, or there may be a multiplexed connection.

The LCD cover plate 122 of the display is transparent. This allows illuminations in the LCD fill 120 to be seen by a user of the device 100. The LCD cover plate 122 may for example be made of a glass material. The display 106 may permanently display a unit in which the measurement is expressed, for example mg/dl, mmol/l or mol/l as shown in FIG. 2. This unit indication may be printed or otherwise adhered to the LCD cover plate 122.

The device 100 is preferably small in size, for example about 10-20 mm wide, 40-80 mm long and less than 10 mm thick. This makes the device 100 easily portable for a user. A user may easily carry a number of these devices 100 around with them. Because the functions of a test strip and analysis device are combined in a single device of small size, a user does not need to carry a separate meter, which may be a relatively bulky item. Multiple devices may be provided to a potential user in individual blister packaging or in a resealable vial. Such packaging keeps the devices 100 and in particular the absorbent material 104 dry and free from contaminants allowing a “shelf life” to be attributed to each device 100.

In operation, a user of the device 100 presents bodily fluid to the exposed end of the absorbent material 104. The bodily fluid is absorbed into the absorbent material 104. The analyte in the bodily fluid undergoes a reaction with a chemical substance present in the absorbent material 104 or undergoes a reaction which is catalyzed by an enzyme present in the absorbent material 104. This reaction produces an electrical charge which may pass as a current through the electrodes 112, 114 and via the conductive paths to the controller 108. The amount of analyte contained in the bodily fluid determines the magnitude of the electrical signal that reaches the controller 108. The property of the electrical signal measured at the controller 108 may be a current and/or voltage of the signal and may involve a time element, for instance by integrating an electrical parameter over time.

As previously described, in some embodiments the controller 108 and display 106 are not activated until an electrical signal above a certain threshold is received via the activation electrodes 114. As more bodily fluid is absorbed, more individual chemical reaction takes place, which increases the amount of electrical charge produced. The activation electrodes 114 are located further from the exposed end of the absorbent material 104 than the measurement electrodes. This ensures that when the threshold signal value is exceeded, there is sufficient quantity of bodily fluid in the volume between the measurement electrodes 112. By activating the controller 108 and display 106 only when a threshold signal level is exceeded, it can be ensured that a sufficient volume of fluid is present in the absorbent material 104 for an accurate measurement to be made.

Once the controller 108 has been activated, it executes software and/or algorithms with which it has been programmed. The controller 108 is programmed to interpret signals received via the measurement electrodes 112 so as to make a measurement of a property of the bodily fluid. The controller 108 may produce a measurement based on a single signal sample received via the measurement electrodes 112. Alternatively the controller 108 may record a series of signals received via the measurement electrodes 112. The controller 108 may compute a measurement based on an analysis of the series of received signals.

Once the controller 108 has computed a measurement of a property of the bodily fluid, it sends signals to the display 106 to cause it to display the result of the measurement. The display may show the measurement for a predetermined time or for as long the capacity of the batteries 110 allow.

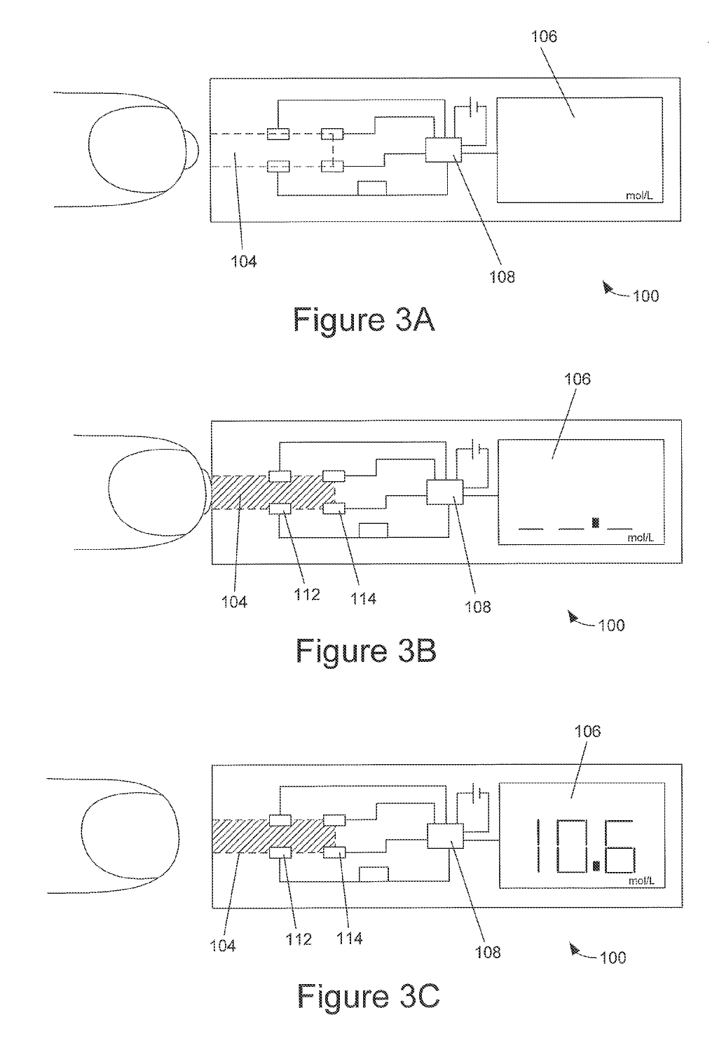

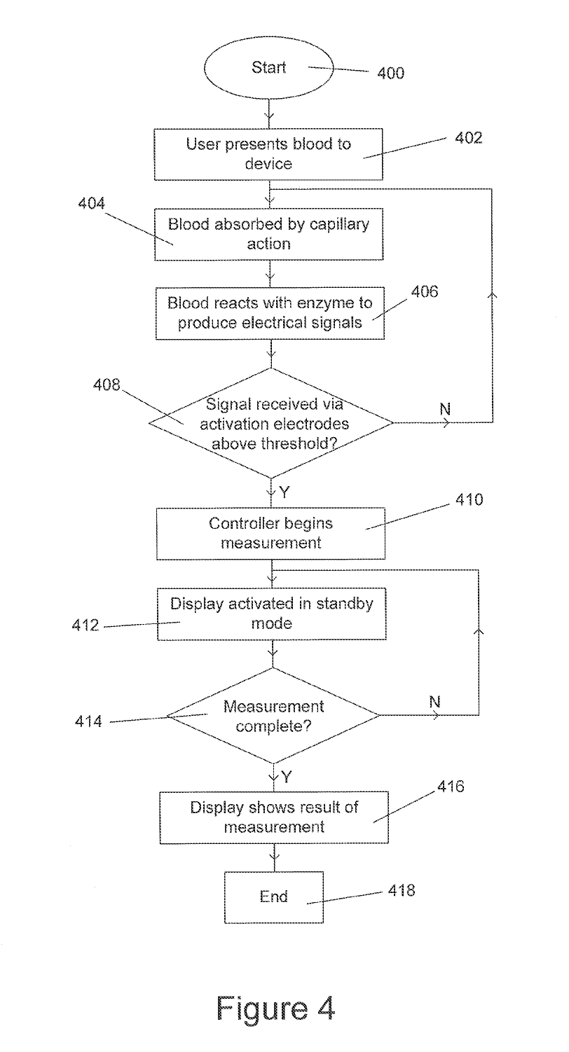

In some embodiments, the bodily fluid which is collected by the device 100 is blood and the property of the blood which is measured is the blood glucose level. The process of making a blood glucose level measurement with the device 100 will now be described with reference to FIGS. 3A-C and the flow chart of FIG. 4.

Referring firstly to FIG. 3A, the device 100 is shown in an idle state. The controller 108 and display 106 are inactive. A user of the device 100 has used a lancet or similar to elicit blood from their finger and is about to present the blood to the exposed end of the absorbent material 104.

In FIG. 3B, the user has presented the blood to the absorbent material 104 by pressing their finger against the exposed end of the absorbent material 104. The blood is absorbed into the absorbent material 104 by capillary action. The absorbent material 104 becomes saturated with the blood as indicated by the cross hatching. The blood reacts with an enzyme present in the absorbent material 104 to produce an electrical current in the absorbent material 104. When an electrical current above a threshold level passes through the activation electrodes 114, the controller 108 and display 106 are activated. The controller 108 begins measuring signals received from the measurement electrodes 112. The display 106 may show a standby state, for example by illuminating the bottom segment of each digit of the display 106.

In FIG. 3C, the controller 108 has finished making its measurement of the blood glucose level of the absorbed blood sample. The controller 108 controls the display 106 to display the result of the measurement to the user. The display 106 continues to display the result of the measurement until the available power supply is exhausted.

FIG. 4 is a flow chart illustrating the process of measuring a blood glucose level using the device 100.

The process begins at step 400. At step 402 the user presents blood to the device 100 at the exposed end of the absorbent material 104. This step of the process is shown in FIG. 3A. At step 404 the blood is absorbed into the device 100 by capillary action. The capillary action causes the blood to fill all of the absorbent material 104 such that it becomes saturated. At step 406 the blood reacts with a chemical substance embedded in the absorbent material 104. Specifically, the glucose in the blood reacts with an enzyme, for example glucose oxidase or glucose dehydrogenase. The reaction produces electrical charge which flows as a current through the saturated absorbent material 104 to the electrodes 112, 114.

At step 408 it is determined if an electrical signal received at the controller 108 via the activation electrodes 114 is above a threshold level. This step may be performed in hardware at the controller 108 such that the controller 108 may remain inactive during the determination. If the signals being received are below the threshold level, the process returns to step 404. This may indicate that not enough blood has been absorbed or that the reaction rate is too low. The device 100 may be configured such that when the absorbent material 104 is saturated with a blood sample of a sufficient quality, the threshold signal value is exceeded. When it is determined that the threshold value is exceeded, the process moves to step 410.

At step 410, the controller 108 begins measuring the blood glucose level in the absorbed blood sample. The controller 108 is configured to receive electrical signals produced by the reaction of the blood glucose with the enzyme in the absorbent material 104 via the measurement electrodes 112. The controller 108 may receive multiple signals in order to make the measurement. The signals may for example be multiple consecutive values of a voltage or a current separated over time. The measurement may involve a time element, for instance integrating the values over time.

At step 412 the display 106 is activated. When initially activated the display 106 may be in a standby mode. This standby mode may consist of illuminating at least some of the pixels of the display. This has the added advantage of signalling to a user of the device 100 that the blood sample they have provided is of a sufficient volume and quality and that a measurement is underway. Step 412 is performed in response to a positive determination at step 408. Step 412 may therefore begin simultaneously with step 410. Step 412 of the process is shown in FIG. 3B.

At step 414 it is determined if the measurement of the blood glucose level is complete. In some embodiments, the controller 108 may perform the measurement relatively quickly based on a single or a few signals from the measurement electrodes 112. In some other embodiments the controller 108 may record signals over a short period of time in order to produce a more accurate measurement of the blood glucose level. If the measurement is not complete, the display continues to operate in standby mode at step 412. Once it is determined that the measurement is complete the process continues at step 416.

At step 416 the display 106 is caused by the controller 108 to display a result of the measurement. The display 106 may continue to display the result of the measurement until the power supply of the device 100 is exhausted. This step is shown in FIG. 3C. In some embodiments, the device 100 is a single use blood glucose meter for taking a single blood glucose level reading. Therefore, after the user has noted the reading from the display 106, the device 100 may be discarded. The process ends at step 418.

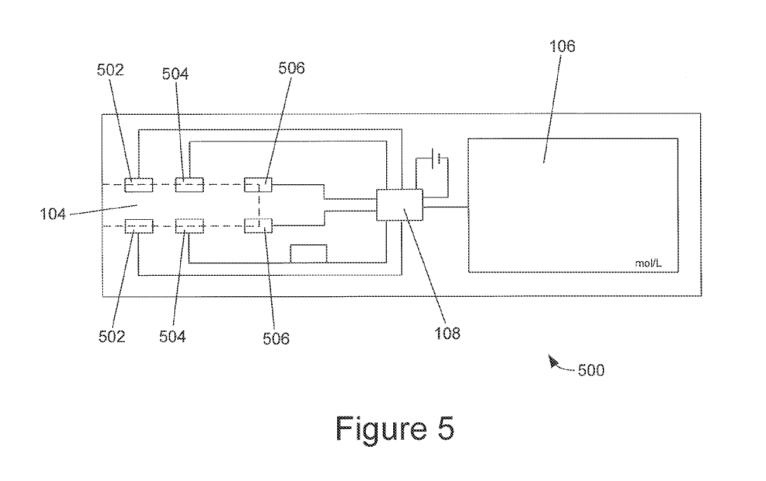

Referring now to FIG. 5, a second embodiment is shown. The device 500 of FIG. 5 differs from the device 100 in that it has three pairs of electrodes. The device 500 has controller activation electrodes 502, measurement electrodes 504 and display activation electrodes 506. Each pair of electrodes 502, 504, 506 is formed on the substrate 102 and overlaps with the absorbent material 104. As shown the controller activation electrodes 502 are located closest to the exposed end of the absorbent material 104. The measurement electrodes 504 are located behind the controller activation electrodes 502; further from the exposed end of the absorbent material 104. The display activation electrodes 506 are located behind the measurement electrodes 504; furthest from the exposed end of the absorbent material 104. All three pairs of electrodes 502, 504, 506 are connected to the controller 108 via conductive paths formed on the substrate 102. The display 106, batteries 110 and resistor 109 of the device 500 are substantially the same as those of the device 100 of the first embodiment and are therefore not described in detail again here.

The controller 108 is configured to receive signals from the controller activation electrodes 502, measurement electrodes 504 and display activation electrodes 506. These signals are produced by the reaction of a bodily fluid sample with a chemical substance or enzyme embedded in the absorbent material 104 as described above in relation to device 100.

The controller 108 of the device 500 may be configured to detect only signals above a certain threshold which are received from the controller activation electrodes 502 and display activation electrodes 506. When a signal above a threshold value is detected via the controller activation electrodes 502, the controller 108 is activated. The controller 108 may then begin recording signals received via the measurement electrodes 504. When a signal above a threshold value is detected via the display activation electrodes 506, the display 106 is activated. The display 106 may be activated in a standby mode. Since the display activation electrodes 506 are located further from the exposed end of the absorbent material 104 than the controller activation electrodes 502, the controller 108 is activated before the display 106. This results in the display 106 being activated for the shortest possible time before a reading is displayed. This reduces the total amount of power required to make a measurement of a property of a bodily fluid and to display the result of the measurement to a user of the device 500 for a given period of time.

The controller 108 and display 106 only draw power from the batteries 110 when active. When inactive, the current discharge from the batteries 110 is zero or close to zero such that the shelf life of the device 100, 500 is not affected.

The signal from display activation electrodes 506 may also be used in the measurement of an analyte in the bodily fluid. For example, by measuring the time when the bodily fluid reaches the display activation electrodes 506, a measurement of the viscosity or the amount of bodily fluid can be made.

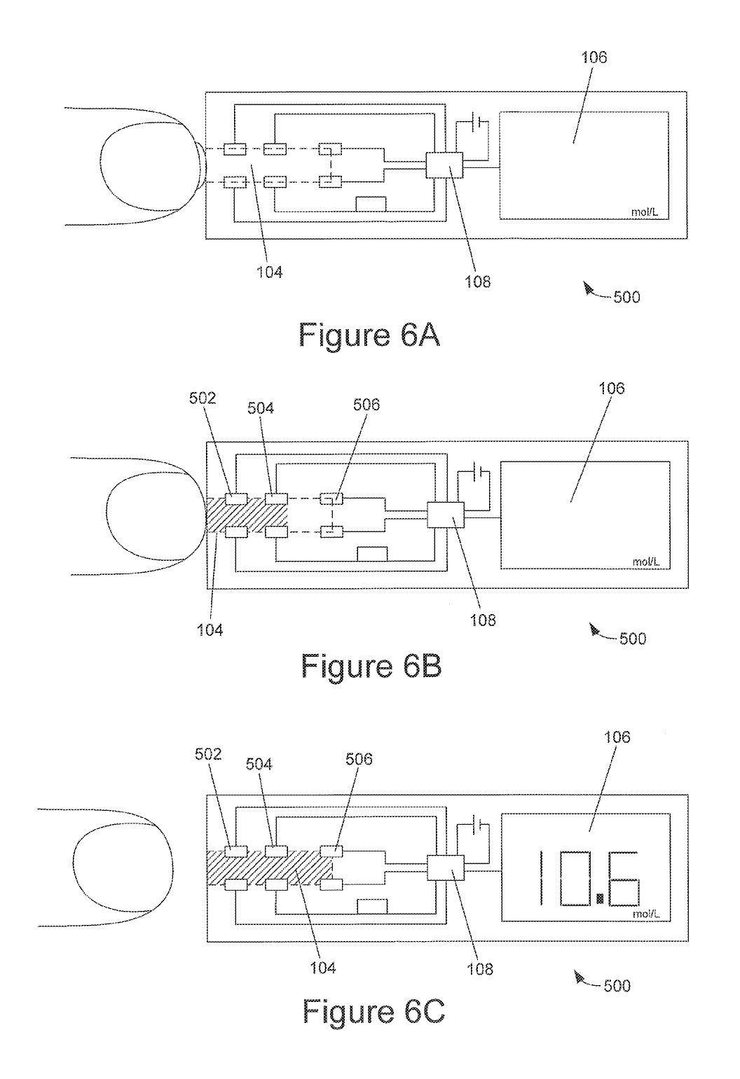

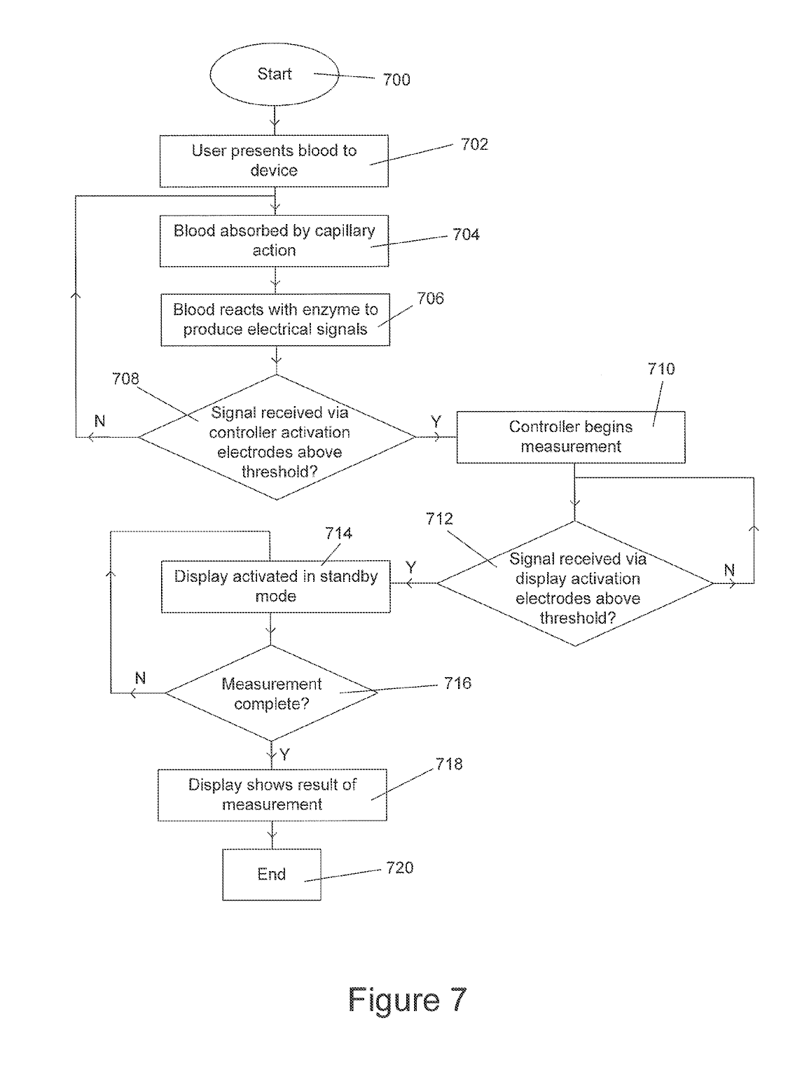

The process of making a blood glucose level measurement with the device 500 will now be described with reference to FIGS. 6A-C and the flow chart of FIG. 7.

In FIG. 6A, the user has presented a blood sample to the absorbent material 104 by pressing their finger against the exposed end of the absorbent material 104. The blood then begins to be absorbed into the absorbent material 104 by capillary action. The controller 108 and display 106 are in an inactive state.

In FIG. 6B, blood is absorbed into the absorbent material 104. The blood reacts with an enzyme present in the absorbent material 104 to produce an electrical current in the absorbent material 104. When an electrical current above a threshold level passes through the controller activation electrodes 502, the controller 108 is activated. The controller 108 then begins measuring signals received from the measurement electrodes 504 in order to compute a blood glucose level of the absorbed blood. The threshold level may be set such that there is a high probability of blood being present between the measurement electrodes 504. At this stage the display 106 remains inactive. Alternatively, only a single segment or symbol of the display 106 is activated to indicate that the device 500 performs a measurement, while all other segments and/or symbols of the display 106 remain inactive.

In FIG. 6C, blood has been absorbed substantially throughout the absorbent material 104. At this stage a threshold signal level passing through the display activation electrodes 506 is exceeded, causing the display 106 to be activated. Once the controller 108 has finished computing the measurement of the blood glucose level, it controls the display 106 to display the result of the measurement. The display 106 continues to display the result of the measurement until the available power supply is exhausted.

FIG. 7 is a flow chart illustrating the process of measuring a blood glucose level using the device 500.

The process begins at step 700. At step 702 the user presents blood to the device 500 at the exposed end of the absorbent material 104. At step 704 the blood is absorbed into the device 500 by capillary action. Step 704 of the process is shown in FIG. 6A.

At step 706 the blood reacts with a chemical substance embedded in the absorbent material 104. Specifically, the glucose in the blood reacts with an enzyme. The reaction produces electrical charge which flows as a current through the saturated absorbent material 104 and out via one of the pairs of electrodes 502, 504, 506 to the controller 108. The amount of glucose in the blood determines the amount of electrical charge created by this reaction.

At step 708 it is determined if an electrical signal received at the controller 108 via the controller activation electrodes 502 is above a threshold level. This step may be performed in hardware at the controller 108 such that the controller 108 may remain inactive during the determination. If the signals being received are below the threshold level, the controller 108 remains inactive and the process returns to step 704, i.e. not enough blood has yet been absorbed or not enough charge has yet been produced by the reaction. When it is determined that the threshold value is exceeded, the process moves to step 710. At this stage it can be assumed that very little charge is flowing via the display activation electrodes 506.

At step 710, the controller 108 begins measuring the blood glucose level in the absorbed blood sample. The controller 108 is configured to receive electrical signals produced by the reaction of the blood glucose with the enzyme in the absorbent material 104 via the measurement electrodes 504. The controller 108 may receive multiple signals in order to make the measurement. Step 710 of the process is shown in FIG. 6B.

At step 712 it is determined if an electrical signal received at the controller 108 via the display activation electrodes 506 is above a threshold level. This step may be performed in hardware or software at the controller 108. If the signals being received are below the threshold level, the display 106 remains inactive. When it is determined that the threshold value is exceeded, the process moves to step 714. The device 500 is configured such that when the absorbent material 104 is saturated with blood, the signal received at the controller 108 via the display activation electrodes 506 exceeds the threshold value.

At step 714 the display 106 is activated. When initially activated the display 106 may be in a standby mode. This standby mode may consist of illuminating at least some of the pixels of the display. This has the added advantage of signalling to a user of the device 500 that the blood sample they have provided is of sufficient volume and quality and that a measurement is underway.

At step 716 it is determined if the measurement of the blood glucose level is complete. If the measurement is not complete, the display 106 continues to operate in standby mode at step 714. Once it is determined that the measurement is complete the process continues at step 718. At some time before it is determined that the signal received via the display activation electrodes 506 exceeds the threshold value, the controller 108 may complete its calculation of the blood glucose level. The controller 108 may then be configured to store the result of the measurement until the display 106 is active or the controller 108 may begin sending control signals to the display 106 immediately on completion of the measurement.

At step 718 the display 106 is caused by the controller 108 to display a result of the measurement. In the situations where the controller 108 has completed its calculation of the blood glucose level before the display is activated, when a positive determination is made at step 712, steps 714 and 716 may occur very quickly such that the display 106 spends only a very brief time in standby mode. This further reduces the power consumption of the device 500. The display 106 may continue to display the result of the measurement until the power supply of the device 500 is exhausted. Step 718 is shown in FIG. 6C. In some embodiments, the device 500 is a single use blood glucose meter for taking a single blood glucose level reading. Therefore, after the user has noted the reading from the display 106, the device 500 may be discarded. The process ends at step 720.

As previously mentioned, the volume of bodily fluid absorbed by the absorbent material 104 may, in some measurement techniques and for some target substances, affect the measurement of that target substance. The absorbent material 104 may therefore be manufactured to absorb a predetermined amount of fluid. This amount may depend on the structure and number of capillaries which form the absorbent material 104. The concentration of chemical substance embedded in the absorbent material 104 and which reacts with the target substance in the bodily fluid may also affect the measurement of the target substance. Although efforts may be made to ensure that each device 100, 500 is identical and each piece of absorbent material 104 used in these devices 100, 500 is identical, there may be some batch variation in the absorptive capacity and chemical substance concentration within the absorbent material 104, particularly when the devices are mass produced. Further, variation in the geometry due to manufacturing tolerances may effect the measurement.

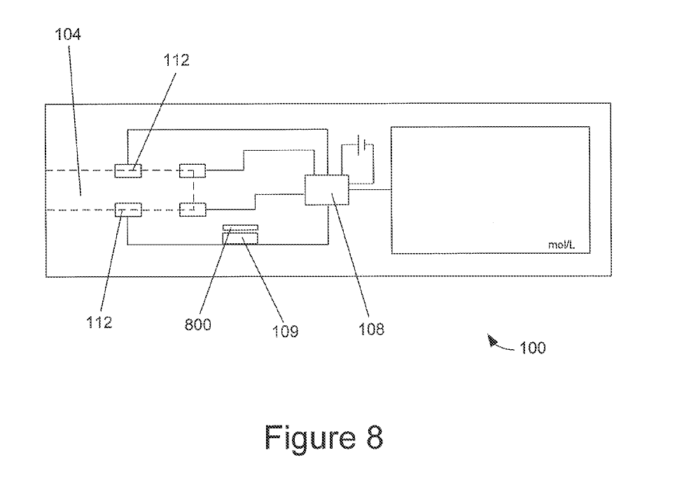

FIG. 8 shows an embodiment of the device 100 in which some batch variation of the absorbent material 104 has occurred during manufacture. For example, one device 100 of each batch may be tested after it has been manufactured in order to determine a calibration value for the devices produced in that batch. In order for the device 100 to be able to produce an accurate measurement of a property of absorbed bodily fluid, the electrical resistance of the measurement circuit path may need to be accurate within a small tolerance. This may be achieved by measuring a control solution with an accurate concentration of analyte, such as glucose.

In the embodiment of the device 100 shown in FIG. 8, the resistor 109 is a printed resistor. Printed resistors can be trimmed with a laser such that their resistance is decreased. After the laser trimming a detached part 800 of resistor 109 no longer forms a part of the measurement circuit path. In this manner, batch variation in the properties of the absorbent material 104 can be corrected.

It will be appreciated that the above described embodiments are purely illustrative and are not limiting on the scope of the invention. Other variations and modifications will be apparent to persons skilled in the art upon reading the present application. Moreover, the disclosure of the present application should be understood to include any novel features or any novel combination of features either explicitly or implicitly disclosed herein or any generalization thereof and during the prosecution of the present application or of any application derived therefrom, new claims may be formulated to cover any such features and/or combination of such features.

1-10. (canceled)

11. An apparatus for analysing a bodily fluid sample, comprising a substrate having supported thereon:
bodily fluid collecting means configured to collect a bodily fluid from the surface of a body part placed adjacent to the bodily fluid collecting means;
means for analysing the collected bodily fluid sample comprising a controller and at least two pairs of electrodes connected to the controller, wherein the at least two pairs of electrodes are integral with the means for collecting a bodily fluid sample and wherein the controller is configured to analyse at least one electrical signal received via a first pair of the at least two pairs of electrodes; and
means for displaying an indication of a result of the analysis,
wherein the apparatus is configured to activate the controller in response to a predetermined electrical signal received via a second pair of the at least two pairs of electrodes.

12. An apparatus according to claim 11, wherein the analysis comprises measuring a property of the bodily fluid and wherein the displaying comprises displaying the result of the measuring.

13. An apparatus according to claim 11, wherein the apparatus is configured to activate the means for displaying in response to a predetermined electrical signal received via the second pair of electrodes.

14. An apparatus according to claim 11, wherein the apparatus is configured to activate the means for displaying in response to a predetermined electrical signal received via a third pair of the at least two pairs of electrodes.

15. An apparatus according to claim 11, wherein the second pair of electrodes is located further from a bodily fluid collecting end of the means for collecting than the first pair of electrodes.

16. An apparatus according to claim 14, wherein the third pair of electrodes is located closer to a bodily fluid collecting end of the means for collecting than the first pair of electrodes.

17. An apparatus according to claim 11, wherein the apparatus further comprises an adjustable calibration resistor.

18. An apparatus according to claim 11, wherein the bodily fluid collecting means comprise an absorbent material.

19. A method for analysing a bodily fluid sample, comprising:
collecting a bodily fluid from a surface of a body part placed adjacent to bodily fluid collecting means;
detecting a predetermined electrical signal received via a second pair of at least two pairs of electrodes integral with the bodily fluid collecting means;
in response to detecting the predetermined signal, activating a controller and analysing the collected bodily fluid sample using the controller by analysing at least one electrical signal received via a first pair of the at least two pairs of electrodes; and
displaying an indication of a result of the analysis.

20. An apparatus, the apparatus comprising:
a substrate, the substrate having supported thereon:
a collector for collecting a bodily fluid from the surface of a body part placed adjacent to the collector;
an analyser for analysing the collected bodily fluid sample, the analyser comprising a controller and at least two pairs of electrodes connected to the controller, wherein the at least two pairs of electrodes are integral with the collector and wherein the controller is configured to analyse at least one electrical signal received via a first pair of the at least two pairs of electrodes; and
a display for displaying an indication of a result of the analysis,
wherein the apparatus is configured to activate the controller in response to a predetermined electrical signal received via a second pair of the at least two pairs of electrodes.
